(19) United States
(12) Patent Application Publication
(10) Pub. No.: US 2019/0053985 A1
(43) Pub. Date: Feb. 21, 2019

(54) EXPIRATION DATE INDICATOR FOR HYPODERMIC NEEDLE DEVICES

(71) Applicant: QUALCOMM Incorporated, San Diego, CA (US)

(72) Inventors: Brian NIZNIK, San Diego, CA (US); Robert GANTON, San Diego, CA (US)

(21) Appl. No.: 16/104,067

(22) Filed: Aug. 16, 2018

Related U.S. Application Data
(60) Provisional application No. 62/547,062, filed on Aug. 17, 2017.

(51) Int. Cl. A61J 7/04

(52) U.S. Cl. CPC A61J 7/0418; A61J 2200/72; A61J 2205/20; A61J 7/0472

(57) ABSTRACT

Disclosed are hypodermic needle devices having an easily-observed, visual indication that the drug contents of the device have expired. The visual indication may be triggered when a counter maintained by a processor of the hypodermic needle device has exceeded a time threshold. The time threshold may be configured based on the drug threshold, and the counter and time threshold may be manually or dynamically adjusted as real-world conditions change to alter the time to expiration. For instance, an prolonged increase in temperature detected by the temperature sensor may speed up the expiration rate of the drug and require a reduction in the time threshold.

### CROSS-REFERENCE TO RELATED APPLICATIONS

This application claims the benefit of U.S. Provisional Application No. 62/547,062, filed Aug. 17, 2017, entitled “EXPIRATION DATE INDICATOR FOR HYPODERMIC NEEDLE DEVICES” which is incorporated herein by reference in its entirety.

### BACKGROUND

### Background Field

The subject matter disclosed herein relates to hypodermic needle devices (e.g., a syringe, an injector pen, a hypodermic needle, etc.), and more particularly to methods and apparatuses for providing a easily-observed, visual indication that the contents of the hypodermic needle device (e.g., a medication) have expired.

### Relevant Background

In the field of drug administration, there are situations where a drug is self-administered by the patient rather than by a healthcare provider. For instance, patients may sometimes self-administer drugs by injecting themselves using simple devices (e.g., hypodermic needles). For simplicity and ease of use, those devices may come pre-loaded with the drug. However, over time the pre-loaded drug may expire and become ineffective or even dangerous when injected into the patient. The devices themselves may lack the capabilities for determining when the drug contents have expired and notifying the patient of expiry. This can easily lead to the patient injecting themselves with the expired drug.

### SUMMARY

Embodiments disclosed herein are directed to hypodermic needle devices that have a processor with a counter, as well as methods of using those hypodermic needle devices. Once the processor is initially switched on, the processor measures the counter against a time threshold. Once the counter exceeds the time threshold, the processor triggers a visual indicator on the device to indicate that the drug contents of the device have expired. In some embodiemnts, the visual indicator may be located somewhere on the device that enables it to be easily spotted by the patient, and the visual indicator may also be configured to be easily interpreted by the patient such that it may be quickly determined whether the drug contents have expired.

Embodiments disclosed herein are also directed to methods of determining drug expiration with a hypodermic needle device. In some embodiments, the method may include initiating a counter, by a processor of the hypodermic needle device. In some embodiments, the method may include determining, by the processor, a time threshold stored in a memory of the hypodermic needle device. In some embodiments, the method may include comparing, by the processor, the counter to the time threshold at pre-programmed intervals. In some embodiments, the method may include triggering, by the processor, an expiration visual element of the hypodermic needle device upon determining that the counter exceeds the time threshold. The expiration visual element may be configured to provide an indication that a drug contained in the hypodermic needle device has expired.

In various embodiments, the expiration visual element is an electro-chromic element. In various embodiments, the expiration visual element is a display screen. In various embodiments, the expiration visual element is a light. In various embodiments, the method may include receiving, from a device separate from the hypodermic needle device, an adjustment to the counter. In various embodiments, the method may include updating, by the processor, the counter based on the adjustment to the counter. In various embodiments, the method may include determining, from a temperature sensor, a change in temperature condition surrounding the hypodermic needle device. In various embodiments, the method may include adjusting, by the processor, the time threshold based on the change in temperature condition.

In various embodiments, the method may include receiving, from a device separate from the hypodermic needle device, an adjustment to the time threshold. In various embodiments, the method may include updating, by the processor, the time threshold based on the adjustment to the time threshold. In various embodiments, the method may include storing, by the processor, the counter in the memory of the hypodermic needle device. In various embodiments, the method may further include determining, from a temperature sensor, a change in temperature condition surrounding the hypodermic needle device. In various embodiments, the method may include sending, to a device separate from the hypodermic needle device, the change in temperature condition. In various embodiments, the method may include receiving, from the device separate from the hypodermic needle device, an adjustment to the time threshold. In various embodiments, the method may include updating, by the processor, the time threshold based on the adjustment to the time threshold. In various embodiments, the method may further include sending a communication to a device separate from the hypodermic needle device. The communication may indicate a value of the counter.

Embodiments disclosed herein are also directed to a hypodermic needle device. In some embodiments, the hypodermic needle device may include a processor. In some embodiments, the hypodermic needle device may include a drug contained in a medicine cartridge. In some embodiments, the hypodermic needle device may include an expiration visual element configured to indicate to a user of the hypodermic needle device that the drug has expired. In some embodiments, the hypodermic needle device may include a memory that may store a set of instructions. In some embodiments, when the set of instructions is executed by the processor, it may cause the processor to initiate a counter. In some embodiments, when the set of instructions is executed by the processor, it may cause the processor to determine a time threshold stored in the memory. In some embodiments, when the set of instructions is executed by the processor, it may cause the processor to compare the counter to the time threshold at pre-programmed intervals. In some embodiments, when the set of instructions is executed by the processor, it may cause the processor to trigger the expiration visual element to provide an indication that the drug has expired, upon determining that the counter exceeds the time threshold.

In various embodiments, the expiration visual element is an electro-chromic element. In various embodiments, the expiration visual element is a display screen. In various embodiments, the expiration visual element is a light. In various embodiments, when the set of instructions is executed by the processor, it may cause the processor to receive, from a device separate from the hypodermic needle device, an adjustment to the counter. In various embodiments, when the set of instructions is executed by the processor, it may cause the processor to update the counter based on the adjustment to the counter. In various embodiments, the hypodermic needle device may further include a temperature sensor. The set of instructions, when executed by the processor, may further cause the processor to determine, from a temperature sensor, a change in temperature condition surrounding the hypodermic needle device, and adjust the time threshold based on the change in temperature condition. In various embodiments, the set of instructions, when executed by the processor, further cause the processor to receive, from a device separate from the hypodermic needle device, an adjustment to the time threshold, and update the time threshold based on the adjustment to the time threshold. In various embodiments, the set of instructions, when executed by the processor, further cause the processor to store the counter in the memory of the hypodermic needle device. In various embodiments, the hypodermic needle device may further include a temperature sensor. The set of instructions, when executed by the processor, may further cause the processor to determine, from the temperature sensor, a change in temperature condition surrounding the hypodermic needle device. The set of instructions, when executed by the processor, may further cause the processor to send, to a device separate from the hypodermic needle device, the change in temperature condition. The set of instructions, when executed by the processor, may further cause the processor to receive, from the device separate from the hypodermic needle device, an adjustment to the time threshold. The set of instructions, when executed by the processor, may further cause the processor to update the time threshold based on the adjustment to the time threshold. In various embodiments, the hypodermic needle device further includes a temperature sensor. The set of instructions, when executed by the processor, may further cause the processor to send a communication to a device separate from the hypodermic needle device. The communication may indicate a value of the counter.

Embodiments disclosed herein are also directed to a hypodermic needle device. In some embodiments, the hypodermic needle device may include means for initiating a counter. In some embodiments, the hypodermic needle device may include means for determining a time threshold stored in a memory of the hypodermic needle device. In some embodiments, the hypodermic needle device may include means for comparing the counter to the time threshold at pre-programmed intervals. In some embodiments, the hypodermic needle device may include means for triggering an expiration visual element of the hypodermic needle device, upon determining that the counter exceeds the time threshold. The expiration visual element may be configured to provide an indication that a drug contained in the hypodermic needle device has expired.

In various embodiments, the expiration visual element is an electro-chromic element. In various embodiments, the expiration visual element is a display screen. In various embodiments, the expiration visual element is a light. In various embodiments, the hypodermic needle device may include means to receive, from a device separate from the hypodermic needle device, an adjustment to the counter. In various embodiments, the hypodermic needle device may include means to update the counter based on the adjustment to the counter. In various embodiments, the hypodermic needle device may include means to determine a change in temperature condition surrounding the hypodermic needle device. In various embodiments, the hypodermic needle device may include means to adjust the time threshold based on the change in temperature condition. In various embodiments, the hypodermic needle device may include means to receive, from a device separate from the hypodermic needle device, an adjustment to the time threshold. In various embodiments, the hypodermic needle device may include means to update the time threshold based on the adjustment to the time threshold.

Embodiments disclosed herein are also directed to a non-transitory computer-readable medium having instructions embedded thereon comprising computer code. In some embodiments, the computer code may instruct a processor of a hypodermic needle device to initiate a counter. In some embodiments, the computer code may instruct a processor of a hypodermic needle device to determine a time threshold stored in a memory of the hypodermic needle device the computer code may instruct a processor of a hypodermic needle device to compare the counter to the time threshold at pre-programmed intervals. the computer code may instruct a processor of a hypodermic needle device to trigger an expiration visual element of the hypodermic needle device upon determining that the counter exceeds the time threshold. The expiration visual element may be configured to provide an indication that a drug contained in the hypodermic needle device has expired.

In various embodiments, the expiration visual element is an electro-chromic element, a display screen, or a light. In various embodiments, the computer code may be further configured to instruct the processor to receive, from a device separate from the hypodermic needle device, an adjustment to the counter, and update the counter based on the adjustment to the counter.

### BRIEF DESCRIPTION OF DRAWINGS

Non-limiting and non-exhaustive aspects are described with reference to the following figures, wherein like reference numerals refer to like parts throughout the various figures unless otherwise specified.

FIG. 1 is an example system for providing information about the administration of medicine by a hypodermic needle device to one or more stakeholders.

FIG. 2 is an illustration of an example hypodermic needle device, according to an embodiment.

FIG. 3 is a block diagram illustrating the of components of a hypodermic needle device, according to an embodiment.

FIG. 4 is a flow diagram illustrating a method used by a hypodermic needle device to determine drug expiration, according to an embodiment.

### DETAILED DESCRIPTION

Several illustrative embodiments will now be described with respect to the accompanying drawings, which form a part hereof. The ensuing description provides embodiment(s) only, and is not intended to limit the scope, applicability, or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

Hypodermic needle devices (e.g., injector pens, auto injectors, syringe needles, etc.) dispense liquid drugs into the body of a patient (e.g., delivering the drug directly into a muscle, vein, or other location under a patient's skin) by pushing a volume of the drug, from the chamber or cylinder within the device, through a hypodermic needle that has been injected into the skin of patient. Oftentimes these drugs may be self-administered by the patient, such as when the drugs are administered in an emergency (as may be the case with epinephrine, for example) or frequently administered (as may be the case with insulin, for example).

Establishing that the right dose of the right drug is dispensed to the right patient at the right time via the right route may be important not only to the person taking the drug, but to many other entities as well. Other stakeholders that have an interest in this information include, for example, the doctor that prescribed and/or is overseeing the administration of the drug, a manufacturer of the drug, an insurance provider (and/or other payer), a government health agency and/or other health organization, and/or the like. For each of the stakeholders, the use and/or misuse of a drug may impact the decisions of a stakeholder with regard to the drug. For example, a drug that is consistently misused may impact how an insurance provider is willing to pay for the use of the drug, and/or how the insurance adjusts premiums for patients that may consistently misuse drugs. It may also prompt a manufacturer to modify the means of administration of the drug to help reduce the misuse of the drug. All stakeholders may be impacted by use/misuse information in determining how effective a drug for a population of patients.

Embodiments of the devices disclosed help supplement the information provided to the stakeholders by notifying the stakeholders when the drug contents of the device has expired. This information may be used and/or combined with other information to help determine whether to administer the drug, to determine whether the drug has been administered properly, and/or to ensure patients are not self-administering expired drugs. Additional details are provided herein below.

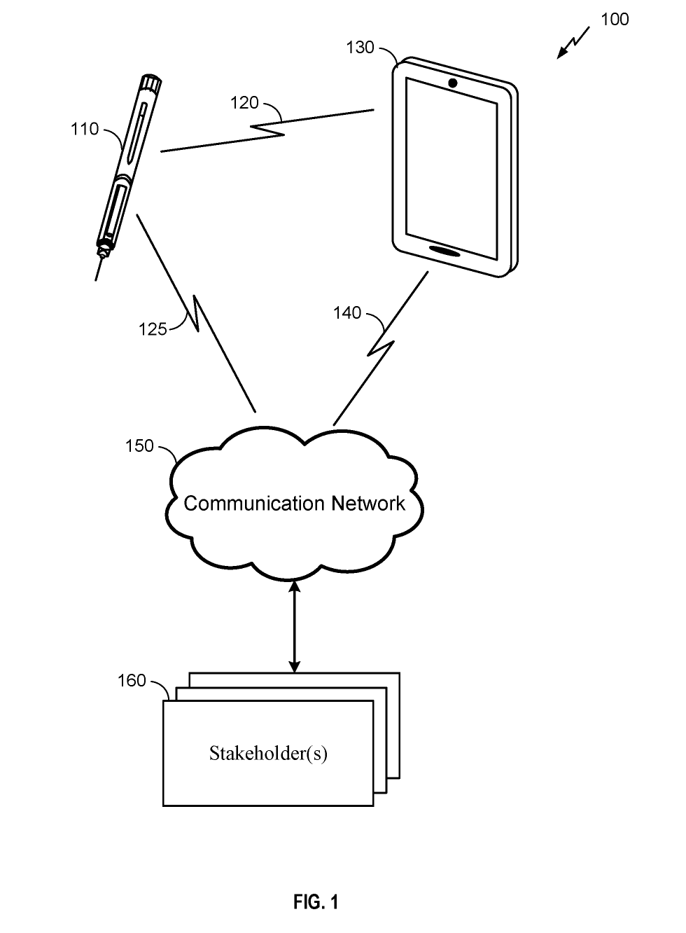

FIG. 1 is an example system 100 for providing information about the administration of medicine by a hypodermic needle device 110 to one or more stakeholders 160. Here, the system 100 may comprise the hypodermic needle device 110 as described herein, along with a connecting device 130, communication network 150, and the stakeholder(s) 160. It will be understood, however, that embodiments of a system 100 may include a different configuration of components, the addition and/or omission of various components, and/or the like, depending on desired functionality. Moreover, it will be understood that techniques described herein may be utilized in a hypodermic needle device 110 that may not necessarily be part of a larger system, such as the system 100 illustrated in FIG. 1.

The hypodermic needle device 110, which is described in more detail herein below, is used to dispense a drug to a patient. Here, a person (e.g., a doctor, a nurse, or a patient) may dispense the drug by engaging a physical mechanism (e.g., pressing down on a plunger, actuating automatic injection, etc.) while a needle of the hypodermic needle device 110 is injected into the patient's skin. In some embodiments, once the drug is dispensed, the hypodermic needle device 110 may register, store and transmit data associated with the administration of the drug to the connecting device 130. This data may be transmitted wirelessly via a wireless communication link 120, using any of a variety of wireless technologies as described in further detail below. That said, some embodiments may additionally, or alternatively, utilize wired communication.

The connecting device 130 may comprise any of a variety of electronic devices capable of receiving information from the hypodermic needle device 110 and communicating information to the stakeholder(s) 160 via the communication network 150. This may include, for example, a mobile phone, tablet, laptop, portable media player, personal computer, or similar device. In some embodiments, the connecting device 130 may comprise a specialized device utilized for purposes of conveying information from the hypodermic needle device 110 (and possibly other medical devices) to the stakeholder(s) 160.

The connecting device 130 may execute an application to provide the data processing and/or relaying functionality illustrated in FIG. 1. In some embodiments, the application may be configurable by a user, or may simply be downloaded to the connecting device 130 and executed automatically. The application may help establish the communication link 120 between the hypodermic needle device 110 and the connecting device 130, which may or may not require input from the user, depending on desired functionality. In some embodiments, the application may provide instructions to a user on proper use of the hypodermic needle device 110 and/or feedback to a user when improper use of the hypodermic needle device 110 is detected. Additional and/or alternative functionality of an application executed by the connecting device 130 may be utilized as desired. (Such functionality may include simple relaying of the data to a remote destination or interacting with the patient about the drug administration, such as providing administration confirmation and handling user feedback.)

The communication network 150 may comprise any of a variety of data communication networks, depending on desired functionality. The communication network 150 may include any combination of radio frequency (RF), optical fiber, satellite, and/or other wireless and/or wired communication technologies. In some embodiments, the communication network 150 may comprise the Internet and/or different data networks may comprise various network types, including cellular networks, Wi-Fi® networks, etc. These types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (Cat-M) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

The communication link 140 between the connecting device 130 and the communication network 150 may vary, depending on the technologies utilized by these components of the system 100. For embodiments where the connecting device 130 is a mobile phone, for example, the communication link 140 may comprise a wireless communication link utilizing the mobile phone's cellular or Wi-Fi® functionality. In embodiments where the connecting device 130 is a personal computer, the communication link 140 may comprise a wired communication link that accesses the medication network 150 via a cable or digital subscriber line (DSL) modem.

It may be noted that some embodiments may not utilize a connecting device 130 to relay data to the communication network 150. In such embodiments, the hypodermic needle device 110 may connect directly to the communication network 150 (as shown in FIG. 1 by communication link 125, which may be used in addition to or as an alternative to communication link 120). For example, the hypodermic needle device 110 may comprise a Long Term Evolution (LTE) category M (Cat-M) device, NarrowBand IoT (NB-IoT), or other Low Power Wide Area Network (LPWAN). Additionally or alternatively, the hypodermic needle device 110 may comprise wireless technology similar to the corresponding functionality of the connecting device 130 described above. In such embodiments, the communication network may additionally or alternatively comprise a Bluetooth Mesh network (such as CSRMesh), a Wi-Fi network, Zigbee, or WWAN (such as LTE, including Cat-M, or 5G). In some embodiments, the hypodermic needle device 110 may connect both with the communication network 150, via medication link 125, and with the connecting device 130, via the communication link 120. In such embodiments, the connecting device 130 may not need to separately communicate information regarding the hypodermic needle device 110 to stakeholders 160, but instead the hypodermic needle device 110 may communicate this information directly to the stakeholders 160 via the communication network 150.

As noted above, the stakeholder(s) 160 may include any of a variety of entities with an interest in the proper administration of medicine using the hypodermic needle device 110. This may include an individual practitioner (e.g., a doctor or nurse), a hospital, a drug manufacturer, an insurance provider (or other payer), a government agency or other health organization, and/or the like. In some embodiments, the user of the hypodermic needle device 110 (e.g., the patient) may also be a stakeholder 160 to which information regarding the use of the hypodermic needle device 110 is provided. Governmental health regulations and/or legal agreements between the patient and/or the stakeholder(s) 160 may apply to the dissemination of information regarding the administration of a drug using the hypodermic needle device 110 to the stakeholder(s) 160.

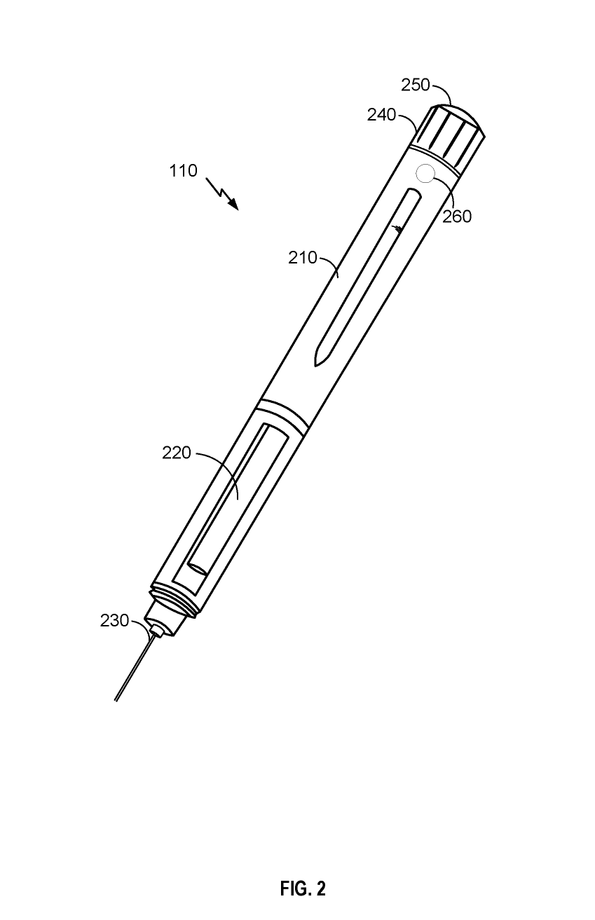

FIG. 2 is an illustration of an example hypodermic needle device 110, according to an embodiment. Here, a body 210 of the hypodermic needle device 110 may house dose dispensing and dose control mechanisms, including electrical and mechanical components, to ensure that a proper dosage of the drug is dispensed. In some embodiments, the components of a dose dispensing mechanism may include a plunger configured to displace a volume of the drug through the reservoir chamber 220 and out the needle assembly 230. Embodiments of a hypodermic needle device 110 may include a dosage dial 240 that may be adjusted (e.g., by turning the dial clockwise or counterclockwise) to alter the dosage amount dispensed by the hypodermic needle device 110. The selected dosage (e.g., set by the dosage dial) may be dispensed by pressing the button 250, which may be coupled to a dose dispensing mechanism to control the dispensing of the drug.

In some embodiments, the hypodermic needle device 110 may have an expiration visual element 260. The expiration visual element 260 may be any suitable component that is capable of indicating, through visual means, that the drug contents of the hypodermic needle device 110 have expired. For example, the expiration visual element 260 could be a display screen, a light (e.g., LED), or an electro-chromic element (e.g., a dot of electro-chromic material that is connected to an electronic module and/or processor that can change colors when triggered). As shown in FIG. 2, the expiration visual element 260 may be an electro-chromic element that may change colors once the drug contents have expired.

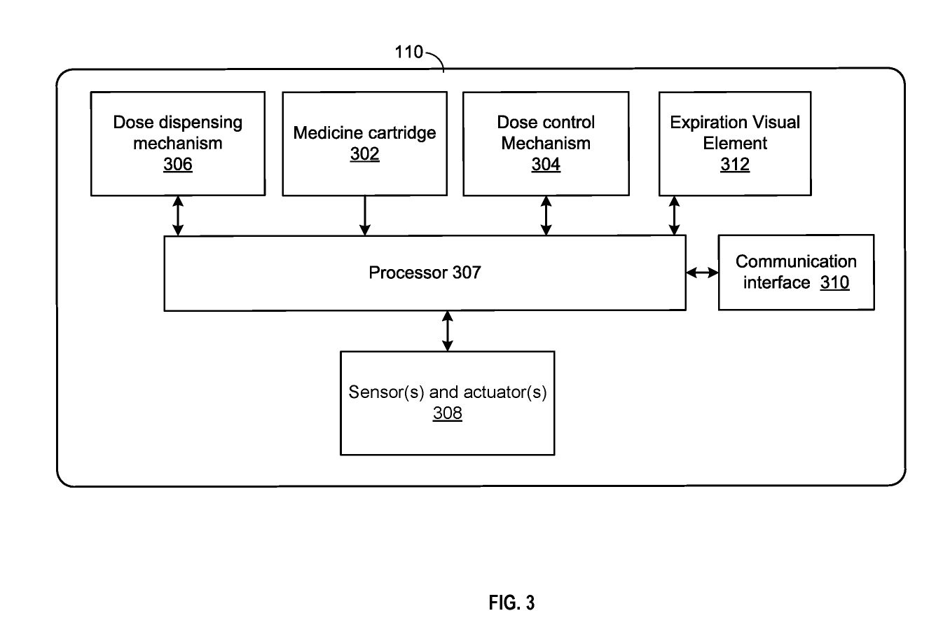

It will be understood however that the hypodermic needle device 110 illustrated in FIG. 2 is provided as a non-limiting example, according to an embodiment. Alternative embodiments may vary in size, shape, and/or other ways. A hypodermic needle device 110 may be described more generally as having various components is illustrated in FIG. 3.

FIG. 3 is a block diagram illustrating the of components of a hypodermic needle device 110, according to an embodiment. The hypodermic needle device 110 may include a housing (not shown) structured to hold a medicine cartridge 302, which may store medicine to be dispensed by the hypodermic needle device 110.

The hypodermic needle device 110 may also include a dose control mechanism 304 to select or set a dose of the drug to be dispensed. For instance, the dose control mechanism 304 may be include a dosage dial (e.g., the dosage dial 240) that may be adjusted to alter the dosage amount dispensed by the hypodermic needle device 110. The hypodermic needle device 110 further includes a dose dispensing mechanism 306 to dispense a dose of the drug (e.g., from the medicine cartridge 302), based on the dose selected or set by dose control mechanism 304.

The hypodermic needle device 110 may also include an expiration visual element 312. The expiration visual element 312 may be any suitable component that is capable of notifying a user through visual means that the drug contents of the hypodermic needle device 110 have expired. For example, the expiration visual element 312 could be a display screen or a light (e.g., LED). In some embodiments, the expiration visual element 312 may be an electro-chromic element, which may be a dot of electro-chromic material that is connected to an electronic module and/or processor. When triggered, the electro-chromic element may provide a visual indication that the drug contents of the hypodermic needle device 110 have expired.

The hypodermic needle device 110 may include other devices and components to facilitate dispensing of medicine. In the example of FIG. 3, the hypodermic needle device 110 includes sensor(s) and actuator(s) 308. Additionally, the hypodermic needle device 110 can include a processor 307, which is communicatively coupled with the sensor(s) and actuator(s) 308 and configured to, among other things, control the operations of the actuator(s) based on the information collected by the sensor(s). For example, the sensors of sensor(s) and actuator(s) 308 may collect information of certain physical conditions at, for example, medicine cartridge 302, dose control mechanism 304, and dose dispensing mechanism 306. Based on the collected information, the processor 307 may control the actuators of sensor(s) and actuator(s) 308 to change the operations of the dose control mechanism 304 and/or dose dispensing mechanism 306.

In some embodiments, the sensor(s) and actuator(s) 308 may include a battery and memory that are electronically coupled to the processor. In some embodiments, the processor 307 may have a non-real time clock (e.g., a counter). The first time that the hypodermic needle device 110 is activated and the processor is switched on (via various means, such as a mechanical, optical, or impedance-based switch or trigger), the processor 307 may begin running the counter. At programmed intervals, the processor 307 may measure the counter against a time threshold set in the memory (e.g., a time threshold of 30 days). If the processor 307 determines that the counter exceeds the threshold, the processor 307 may trigger the expiration visual element 312 to provide a visual indication that the drug contents of the hypodermic needle device 110 have expired. In some embodiments, the expiration visual element 312 may be an electro-chromic element (e.g., a dot). The processor 307 may instruct an electronic module associated with the electro-chromic element to activate the electro-chromic element to change the color of the dot (e.g., turning the electro-chromic element from a white dot to a red dot). If the counter does not exceed the time threshold, then the counter continues to run until the processor 307 determines that the counter exceeds the threshold. In some embodiments, once the time threshold has been exceeded, the processor 307 may wirelessly send a message to any stakeholders that the drug has expired (e.g., via the communication link 120, communication link 125, and/or the communication network 150 shown in FIG. 1).

In some embodiments, the time threshold may be added to the memory when the hypodermic needle device 110 is manufactured. In some embodiments, the time threshold in the memory may be provided to the hypodermic needle device 110, such as wirelessly via the communication link 120, communication link 125, and/or the communication network 150 shown in FIG. 1. For instance, one of the stakeholders 160 shown in FIG. 1 may utilize a device, such as connecting device 130, to set the time threshold on the hypodermic needle device 110 based on the expiration date of the drug in the medicine cartridge 302.

In some embodiments, the hypodermic needle device 110 may determine or modify the expiration time threshold based on temperature or temperature variations. The hypodermic needle device 110 may have a temperature sensor (e.g., included in the sensor(s) and actuator(s) 308) that is configured to measure temperature and/or variations in temperature in the drug or the surrounding environment. In practice, the expiration time for a drug may vary depending on the temperature that the drug is stored at. For instance, a drug may have a much longer expiration time when the hypodermic needle device 110 is stored at low temperatures. In contrast, any periods of time where the hypodermic needle device 110 is subjected to higher temperatures may reduce the expiration time for the drug. Thus, the processor 307 of the hypodermic needle device 110 may be able to dynamically calculate and update the time threshold in the memory based on changing temperature conditions. For example, if the temperature measured from the temperature sensor exceeds a certain value, then the processor 307 may decrease the time threshold due to the decrease in the longevity of the drug. In turn, this lowering of the time threshold will reduce the time needed for the counter to be greater than the time threshold, which will mean the expiration visual element 312 will be triggered sooner. Thus, the expiration visual element 312 may be dynamically triggered based on variations in the surrounding temperature. In some embodiments, the processor 307 may check the temperature sensor at pre-programmed intervals in order to continually monitor the temperature for the potential to update the time threshold.

In some embodiments, the temperature variations observed by the hypodermic needle device 110 may be sent from the hypodermic needle device 110 to a separate device, such as a remote computer, mobile device, smartphone, and so forth. For example, the temperature variations may be sent to a computer where it may be viewed by a doctor. The doctor may take that temperature variation into consideration to vary the drug expiration time. The doctor may then use the computer to send an updated time threshold to the hypodermic needle device 110 to replace the existing time threshold stored in the memory. Thus, a user may also have access to the time threshold and be able to manually update it to account for any variations in the expiration time of the drug.

Moreover, the hypodermic needle device 110 may include a communication interface 310 which may communicate using wireless and/or wired means (e.g., via wireless link 120 and/or 125 of FIG. 1). Communication interface 310 may enable transmission of information related to dispensing the drug, including an indication of whether the drug was dispensed properly or an indication that the drug has expired. Additionally or alternatively, the hypodermic needle device 110 may communicate information related to a quantity of medicine to be dispensed, a quantity of medicine that has been dispensed, a quantity of medicine remaining in medicine cartridge 302, etc. The information may then be displayed to the user via an user interface, to assist the user in dispensing of the medicine.

In some embodiments, the expiration visual element 312 may be on a separate device from the hypodermic needle device 110, such as a remote computer, mobile device, smartphone, and so forth. When the counter is greater than the time threshold, the hypodermic needle device 110 may send a communication to that separate device (that has the expiration visual element 312) in order to trigger the expiration visual element 312. In some embodiments, this communication may be sent via the communication interface 310, which may send the communication using wireless and/or wired means. Thus, any description herein regarding an expiration visual element 312 within a hypodermic needle device 110 may be similarly applicable to an expiration visual element 312 implemented in a separate device.

In some embodiments, the value of the counter (e.g., operated by the processor) may be also be reported to a user (e.g., a patient, doctor, or pharmacist) via the communication interface 310. For example, the counter value may be sent to a separate device, such as a remote computer, mobile device, smartphone, and so forth. In some embodiments, the counter may be adjusted. For example, the separate device may provide a user interface that allows the counter value to be updated. The updated counter value may then be sent from the separate device back to the hypodermic needle device 110, and the processor of the hypodermic needle device 110 may update the counter with the received updated counter value. This feature allows a doctor or pharmacist to adjust the counter, which may be useful based on the time of purchase of the drug (e.g., to account for a delay in a patient receiving the drug), or if the drug lot has problems associated with it. The doctor may also be able to send notifications to the user of the hypodermic needle device 110, allowing the doctor to notify the user of potential problems with that drug lot. This notification may be a new flag or an indication that the drug has expired, which will immediately trigger the expiration visual element 312 (e.g., by setting the time threshold to be lower than the counter, or by setting the counter to a value that is greater than the time threshold).

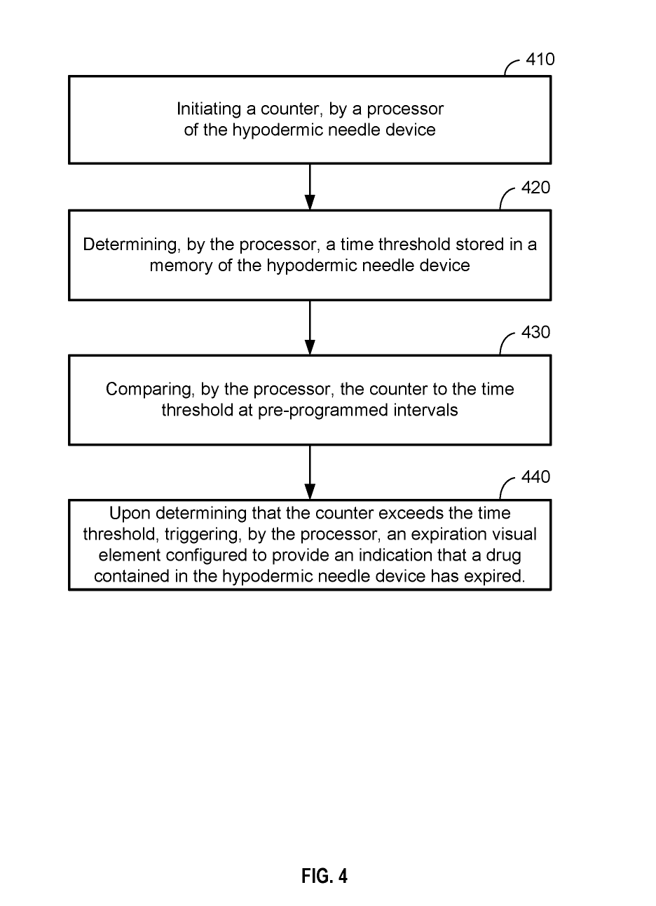

FIG. 4 is a flow diagram illustrating a method used by a hypodermic needle device to determine drug expiration, according to an embodiment.

At block 410, a processor of the hypodermic needle device may be switched on for the first time. The processor will initiate a counter (e.g., a stopwatch or a non-real time clock) that the processor will continue to maintain. The processor may also store the counter values in a memory of the hypodermic needle device in case the battery or power to the device runs out. The processor may then retrieve the previous counter values and continue where it left off when the power is restored.

Means for performing the functionality of block 410 may comprise, for example, a processor, such as processor 307 shown in FIG. 3, initiating a counter. The processor may store the counter value in memory (not shown in FIG. 3).

At block 420, the processor may determine a time threshold stored in the memory of the hypodermic needle device. For example, the time threshold may be 30 days or 48 hours, and so forth. In some embodiments, the time threshold in the memory have by the communication link 120, communication link 125, and/or the communication network 150 shown in FIG. 1. For instance, one of the stakeholders 160 shown in FIG. 1 may utilize a device, such as connecting device 130, to send a wireless communication setting the time threshold on the hypodermic needle device 110 based on the expiration date of the drug in the device.

Means for performing the functionality of block 420 may comprise, for example, a processor, such as processor 307 shown in FIG. 3, determining a time threshold stored in memory. The processor 307 may also perform this function by communicating with other devices via the communication interface 310 illustrated in FIG. 3, as well as the communication link 120 and/or communication network 150 shown in FIG. 1 and described above.

At block 430, the processor may compare the counter to the time threshold at pre-programmed intervals to check if the counter is greater than the time threshold. For instance, the processor may compare the counter to the time threshold every hour, once every day, and so forth. Longer intervals may conserve battery and power, which may be preferable for longer drug expiration times. Shorter intervals use more battery and power since the processor must check more often, but shorter intervals are faster for determining the instant that the counter is greater than the time threshold and may be preferable for shorter drug expiration times. In some embodiments, the interval used may be automatically adjusted based on the time threshold and drug expiration time, such that the device will have sufficient power for the processor to make the comparison over the duration of time that it takes for the drug to expire.

Means for performing the functionality of block 430 may comprise, for example, a processor, such as processor 307 shown in FIG. 3, comparing the counter to the time threshold.

At block 440, upon determining that the counter exceeds the time threshold, the processor may trigger an expiration visual element of the hypodermic needle device to notify a user that the drug contained in the device has expired. For example, the expiration visual element could be a display screen or a light (e.g., LED). The processor may operate the display screen to indicate the drug has expired, or alternatively, the processor may switch on the light. In some embodiments, the expiration visual element may be an electro-chromic element (e.g., a visible dot). The processor may trigger the electro-chromic element to change color, which will provide a visual indication that the drug contents of the hypodermic needle device have expired. In some embodiments, the processor may also send a communication (e.g., wirelessly) to one or more devices indicating that the drug contents of the device have expired. For instance, the stakeholders 160 shown in FIG. 1 may operate those devices receiving the communication and learn that the drug has expired. The stakeholders 160 may then use that information, such as for the purposes of informing the patient not to self-administer the expired drug using the device or to send a medicine cartridge to the patient that contains unexpired drug.

Means for performing the functionality of block 440 may comprise, for example, a processor, such as processor 307 shown in FIG. 3, sending a signal to the expiration visual element 312 shown in FIG. 3.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

With reference to the appended figures, components that may comprise memory may comprise non-transitory machine-readable media. The term “machine-readable medium” and “computer-readable medium” as used herein, refer to any storage medium that participates in providing data that causes a machine to operate in a specific fashion. In embodiments provided hereinabove, various machine-readable media might be involved in providing instructions/code to processing units and/or other device(s) for execution. Additionally or alternatively, the machine-readable media might be used to store and/or carry such instructions/code. In many implementations, a computer-readable medium is a physical and/or tangible storage medium. Such a medium may take many forms, including but not limited to, non-volatile media, volatile media, and transmission media. Common forms of computer-readable media include, for example, magnetic and/or optical media, any other physical medium with patterns of holes, a RAM, a PROM, EPROM, a FLASH-EPROM, any other memory chip or cartridge, a carrier wave as described hereinafter, or any other medium from which a computer may read instructions and/or code.

The methods, systems, and devices discussed herein are examples. Various embodiments may omit, substitute, or add various procedures or components as appropriate. For instance, features described with respect to certain embodiments may be combined in various other embodiments. Different aspects and elements of the embodiments may be combined in a similar manner. The various components of the figures provided herein may be embodied in hardware and/or software. Also, technology evolves and, thus, many of the elements are examples that do not limit the scope of the disclosure to those specific examples.

Reference throughout this specification to “one example”, “an example”, “certain examples”, or “exemplary implementation” means that a particular feature, structure, or characteristic described in connection with the feature and/or example may be included in at least one feature and/or example of claimed subject matter. Thus, the appearances of the phrase “in one example”, “an example”, “in certain examples” or “in certain implementations” or other like phrases in various places throughout this specification are not necessarily all referring to the same feature, example, and/or limitation. Furthermore, the particular features, structures, or characteristics may be combined in one or more examples and/or features.

Some portions of the detailed description included herein are presented in terms of algorithms or symbolic representations of operations on binary digital signals stored within a memory of a specific apparatus or special purpose computing device or platform. In the context of this particular specification, the term specific apparatus or the like includes a general purpose computer once it is programmed to perform particular operations pursuant to instructions from program software. Algorithmic descriptions or symbolic representations are examples of techniques used by those of ordinary skill in the signal processing or related arts to convey the substance of their work to others skilled in the art. An algorithm is here, and generally, is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer, special purpose computing apparatus or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

The terms, “and”, “or”, and “and/or” as used herein may include a variety of meanings that also are expected to depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein may be used to describe any feature, structure, or characteristic in the singular or may be used to describe a plurality or some other combination of features, structures or characteristics. Though, it should be noted that this is merely an illustrative example and claimed subject matter is not limited to this example.

Therefore, it is intended that claimed subject matter not be limited to the particular examples disclosed, but that such claimed subject matter may also include all aspects falling within the scope of appended claims, and equivalents thereof.

What is claimed is:

1. A method of determining drug expiration with a hypodermic needle device, the method comprising:
initiating a counter, by a processor of the hypodermic needle device;
determining, by the processor, a time threshold stored in a memory of the hypodermic needle device;
comparing, by the processor, the counter to the time threshold at pre-programmed intervals; and
upon determining that the counter exceeds the time threshold, triggering, by the processor, an expiration visual element of the hypodermic needle device, wherein the expiration visual element is configured to provide an indication that a drug contained in the hypodermic needle device has expired.

2. The method of claim 1, wherein the expiration visual element is an electro-chromic element.

3. The method of claim 1, wherein the expiration visual element is a display screen.

4. The method of claim 1, wherein the expiration visual element is a light.

5. The method of claim 1, further comprising:
receiving, from a device separate from the hypodermic needle device, an adjustment to the counter; and
updating, by the processor, the counter based on the adjustment to the counter.

6. The method of claim 1, further comprising:
determining, from a temperature sensor, a change in temperature condition surrounding the hypodermic needle device; and
adjusting, by the processor, the time threshold based on the change in temperature condition.

7. The method of claim 1, further comprising:
receiving, from a device separate from the hypodermic needle device, an adjustment to the time threshold; and
updating, by the processor, the time threshold based on the adjustment to the time threshold.

8. The method of claim 1, further comprising:
storing, by the processor, the counter in the memory of the hypodermic needle device.

9. The method of claim 1, further comprising:
determining, from a temperature sensor, a change in temperature condition surrounding the hypodermic needle device;
sending, to a device separate from the hypodermic needle device, the change in temperature condition;
receiving, from the device separate from the hypodermic needle device, an adjustment to the time threshold; and
updating, by the processor, the time threshold based on the adjustment to the time threshold.

10. The method of claim 1, further comprising:
sending a communication to a device separate from the hypodermic needle device, wherein the communication indicates a value of the counter.

11. A hypodermic needle device comprising:
a processor;
a drug contained in a medicine cartridge;
an expiration visual element configured to indicate to a user of the hypodermic needle device that the drug has expired; and
a memory, wherein the memory stores a set of instructions, that when executed by the processor, causes the processor to:
initiate a counter;
determine a time threshold stored in the memory;
compare the counter to the time threshold at pre-programmed intervals; and
upon determining that the counter exceeds the time threshold, trigger the expiration visual element to provide an indication that the drug has expired.

12. The hypodermic needle device of claim 11, wherein the expiration visual element is an electro-chromic element.

13. The hypodermic needle device of claim 11, wherein the expiration visual element is a display screen.

14. The hypodermic needle device of claim 11, wherein the expiration visual element is a light.

15. The hypodermic needle device of claim 11, wherein the set of instructions, when executed by the processor, further cause the processor to:
receive, from a device separate from the hypodermic needle device, an adjustment to the counter; and
update the counter based on the adjustment to the counter.

16. The hypodermic needle device of claim 11, wherein the hypodermic needle device further comprises a temperature sensor, and wherein the set of instructions, when executed by the processor, further cause the processor to:
determine, from a temperature sensor, a change in temperature condition surrounding the hypodermic needle device; and
adjust the time threshold based on the change in temperature condition.

17. The hypodermic needle device of claim 11, wherein the set of instructions, when executed by the processor, further cause the processor to:
receive, from a device separate from the hypodermic needle device, an adjustment to the time threshold; and
update the time threshold based on the adjustment to the time threshold.

18. The hypodermic needle device of claim 11, wherein the set of instructions, when executed by the processor, further cause the processor to:
store the counter in the memory of the hypodermic needle device.

19. The hypodermic needle device of claim 11, wherein the hypodermic needle device further comprises a temperature sensor, and wherein the set of instructions, when executed by the processor, further cause the processor to:
determine, from the temperature sensor, a change in temperature condition surrounding the hypodermic needle device;
send, to a device separate from the hypodermic needle device, the change in temperature condition;
receive, from the device separate from the hypodermic needle device, an adjustment to the time threshold; and
update the time threshold based on the adjustment to the time threshold.

20. The hypodermic needle device of claim 11, wherein the hypodermic needle device further comprises a temperature sensor, and wherein the set of instructions, when executed by the processor, further cause the processor to:
send a communication to a device separate from the hypodermic needle device, wherein the communication indicates a value of the counter.

21. A hypodermic needle device comprising:
means for initiating a counter;
means for determining a time threshold stored in a memory of the hypodermic needle device;
means for comparing the counter to the time threshold at pre-programmed intervals;
means for triggering, upon determining that the counter exceeds the time threshold, an expiration visual element of the hypodermic needle device, wherein the expiration visual element is configured to provide an indication that a drug contained in the hypodermic needle device has expired.

22. The hypodermic needle device of claim 21, wherein the expiration visual element is an electro-chromic element.

23. The hypodermic needle device of claim 21, wherein the expiration visual element is a display screen.

24. The hypodermic needle device of claim 11, wherein the expiration visual element is a light.

25. The hypodermic needle device of claim 21, further comprising:
means to receive, from a device separate from the hypodermic needle device, an adjustment to the counter; and
means to update the counter based on the adjustment to the counter.

26. The hypodermic needle device of claim 21, further comprising:
means to determine a change in temperature condition surrounding the hypodermic needle device; and
means to adjust the time threshold based on the change in temperature condition.

27. The hypodermic needle device of claim 21, further comprising:
means to receive, from a device separate from the hypodermic needle device, an adjustment to the time threshold; and
means to update the time threshold based on the adjustment to the time threshold.

28. A non-transitory computer-readable medium having instructions embedded thereon comprising computer code for instructing a processor of a hypodermic needle device to:
initiate a counter;
determine a time threshold stored in a memory of the hypodermic needle device;
compare the counter to the time threshold at pre-programmed intervals; and
upon determining that the counter exceeds the time threshold, trigger an expiration visual element of the hypodermic needle device, wherein the expiration visual element is configured to provide an indication that a drug contained in the hypodermic needle device has expired.

29. The non-transitory computer-readable medium of claim 28, wherein the expiration visual element is an electro-chromic element, a display screen, or a light.

30. The non-transitory computer-readable medium of claim 28, wherein the computer code is further configured to instruct the processor to:
receive, from a device separate from the hypodermic needle device, an adjustment to the counter; and
update the counter based on the adjustment to the counter.
